(19) United States
(12) Patent Application Publication
(10) Pub. No.: US 2009/0105617 A1
(43) Pub. Date: Apr. 23, 2009

(54) Non-invasive quantitative body contouring by high intensive focused ultrasound

(75) Inventor: Cheng Yi, Marlboro, NJ (US)

Correspondence Address: Cheng Yi, 9 Graversham Drive, Marlboro, NJ, 07746, US

(73) Assignee: PrecisCurve, Inc

(21) Appl. No.: 12/287,405

(22) Filed: Oct. 10, 2008

Related U.S. Application Data
(60) Provisional application No. 60/998,907, filed on Oct. 15, 2007.
(60) Provisional application No. 61/010,037, filed on Jan. 7, 2008.

(51) Int. Cl. A61N 7/00; A61B 18/04

(52) U.S. Cl. 601/2; 606/28

(57) ABSTRACT

An apparatus is invented for breaking adipose tissue involving delivering ultrasonic energy at a multiplicity of target layers of volume under the skin. The delivered ultrasound is focused on the target layers. One or a plurality of ultrasonic focuses scan the target layers. The scanning ultrasonic focuses break the adipose tissue quantitatively, while other non-adipose tissues are generally not broken. The quantitative breaking process of adipose tissue is performed by quantitative feedback information. Another ultrasound of different frequency helps to move the broken adipose tissue away from the targeted volumes in body circulation. An ultrasound transducer assembly has at least one transducer array of high intensity focused ultrasound (HIFU) and at least one transducer of high intensity unfocused ultrasound.

### CROSS-REFERENCES TO RELATED APPLICATIONS

The present application is a non-provisional of U.S. Patent Application Ser. Nos. 60/998,907, filed on Oct. 15, 2007, and 61/010,037, filed on Jan. 7, 2008, the full disclosure of which is incorporated herein by reference.

### FIELD OF THE INVENTION

The present invention relates to an apparatus for breaking adipose tissue with high intensity focused ultrasound.

### BACKGROUND OF THE INVENTION

The following U.S. patents or patent applications are believed to represent the current state of the art: U.S. Pat. Nos. 7,347,855; 6,607,498. U.S. Patent Application No. 2004003912.

There are several medical systems which use extracorporeal high energy ultrasound to destroy or lyse adipose tissue inside the human body.

One method is to apply high intensity focused ultrasound (HIFU) into a target volume inside a human body and cause a high local temperature to boil the adipose tissue. This method is described in U.S. Patent Application 20040039312.

Another method is to apply high energy ultrasound into a target volume within a human body. It does not cause much thermal effect. Instead, it uses mechanical force to rupture the adipose cells. To get this effect the inventors apply either periodic ultrasound or modulated ultrasound to the target volume. This method is described in the U.S. Pat. Nos. 6,607,498 and 7,347,855.

There are two ways to focus ultrasound. One is geometrical. This one changes the direction of ultrasound to focus the ultrasound beam on a spot. Another focusing is through phase-coordination. In the phase-focusing, it usually has multiple ultrasound transducers. The ultrasound coming out of each transducer is phased coordinated to get a maximal pressure at a certain spot, which can also be called a focus. This spot can change when the phase coordination is changed. This phase-focusing is also called electronic focusing.

### SUMMARY OF THE INVENTION

The present invention is directed to ultrasonic breaking of adipose tissue in a way that is substantially different from the relevant art.

The present invention relates to equipment that breaks adipose tissue within a human's body using high intensity focused ultrasound (HIFU), the equipment is comprising:

A central computing sub-system for sending commands and storing and analyzing data and for controlling a plurality of electronic components.

A transducer assembly having at least one transducer array for emitting high intensive focused ultrasound into the human body.

A timing control unit coordinating the driving signals of said elements of transducer arrays to obtain at least one variable ultrasonic focus.

The said transducer assembly also having at least one transducer for emitting a different frequency of high intensive unfocused ultrasound into the human body.

The said transducer assembly also having at least one sensor for detecting some signals' variations caused by the breaking process of adipose tissue and sending back the information of said variations to said central computing sub-system.

A set-up for placing a human body and positioning the target adipose tissue within the said human body, where the said transducer assembly can aim at from outside of said human body.

Advantage of the present invention is to use at least one variable ultrasonic focus to scan a layer of biological tissues in a target volume within said human body. This will significantly reduce the time of patients' treatment. It can also perform accurate body contouring by breaking different thickness of said adipose layers and different depth of said adipose tissue and different amount of said adipose tissue.

Another advantage of the present invention is to quantitative breaking of adipose tissue. The previous art of breaking adipose tissue is just targeting successful rate. This invention is to quantize the amount of adipose tissue has been broken. It can be either relative amount like the percent of said total estimated local adipose tissue or absolute amount. This is done through measurement of backscattered ultrasound. The measurements of said variations will be fed back to said central computing sub-system to map the amount of broken adipose tissue.

Another advantage of the present invention is that the said backscattered ultrasound measurement can also provide the location and size of adipose tissue.

Another advantage of the present invention is to use an unfocused high intensive ultrasound to dilate local blood vessels in said target volume and to accelerate the lymphatic return. This unfocused ultrasound has a different frequency from the focused one. The focused ultrasound has a frequency range from 250 KHz to 1.2 MHz. The unfocused one has a frequency of range from 28 KHz to 250 KHz.

Additional advantages and features of this invention will be stated in the following descriptions and appended drawings.

### BRIEF DESCRIPTION OF THE DRAWINGS

FIG. 1 illustrates how the invented system works.

FIG. 2 illustrates the system diagram of the invention with backscattered ultrasound measurement.

FIGS. 3a and 3b illustrate two kinds of high frequency HIFU transducer array, square shape and circular shape.

FIGS. 4a and 4b illustrate two kinds of low frequency transducer, square shape and circular shape.

FIGS. 5a and 5b illustrate a pair of one high frequency HIFU transducer array and one low frequency ultrasound transducer of matched size stacking-up.

FIGS. 6a and 6b illustrate a pair of four high frequency HIFU transducer arrays and one low frequency ultrasound transducer, stacking-up.

FIGS. 7a and 7b illustrate a pair of one high frequency HIFU transducer array and one low frequency ultrasound transducer, of, smaller size stacking-up.

FIG. 8 illustrates a pair of one high frequency HIFU transducer array and one low frequency ultrasound transducer of smaller size stacking-up. The two transducers have a circular size.

FIG. 9 illustrates how a HIFU transducer array, generates ultrasound beams which scan a layer of volume of biological tissue.

### DETAILED DESCRIPTION OF THE INVENTION

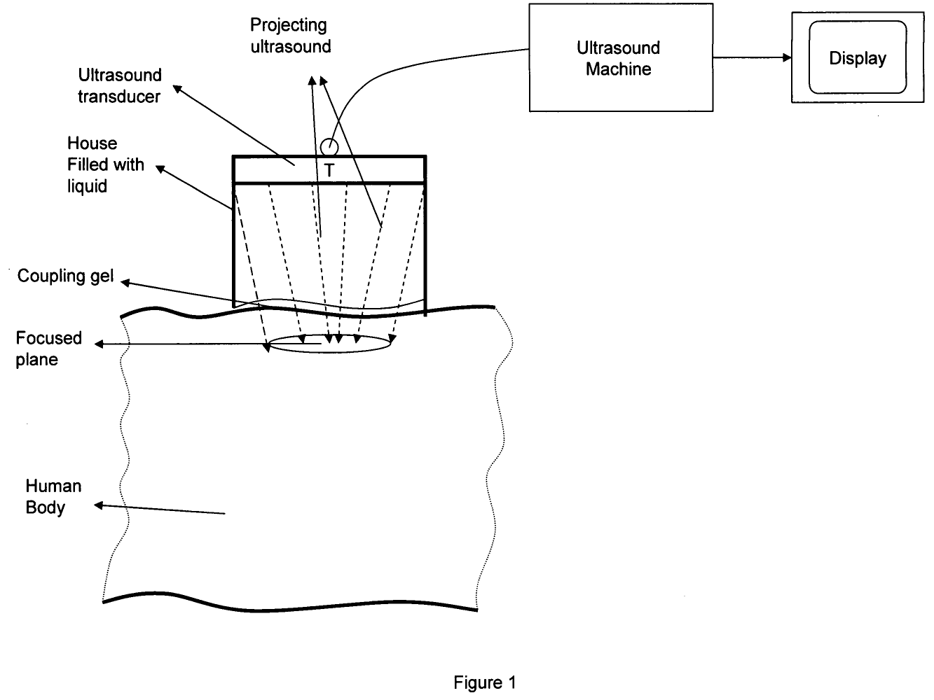

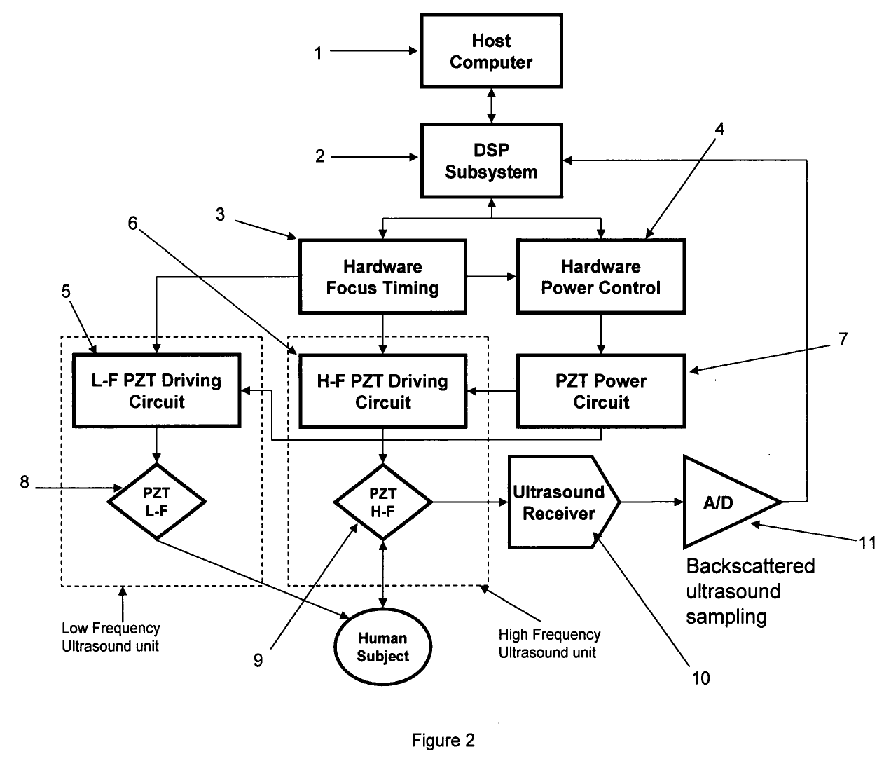

A simplified illustration showing how the invention works is demonstrated in FIGS. 1. The ultrasound transducers sitting in a house filled with degassed liquid in the front. The ultrasound machine is part of this invention, which will control, project and focus the ultrasound into human body. The ultrasonic focus will scan a plane, which is called a focused plane, in the human body. A single focused plane is shown here. It can be multiple ones. When a single or multiple ultrasonic focuses scanning the focused planes, the scanned adipose tissue is eventually broken and non-adipose tissues are intact. The invented system is illustrated in FIG. 2. A host computer 1 or Unit 1 is the interface between the ultrasound system and the operators (doctors). Commands, input data and feedback data are carried through the host computer.

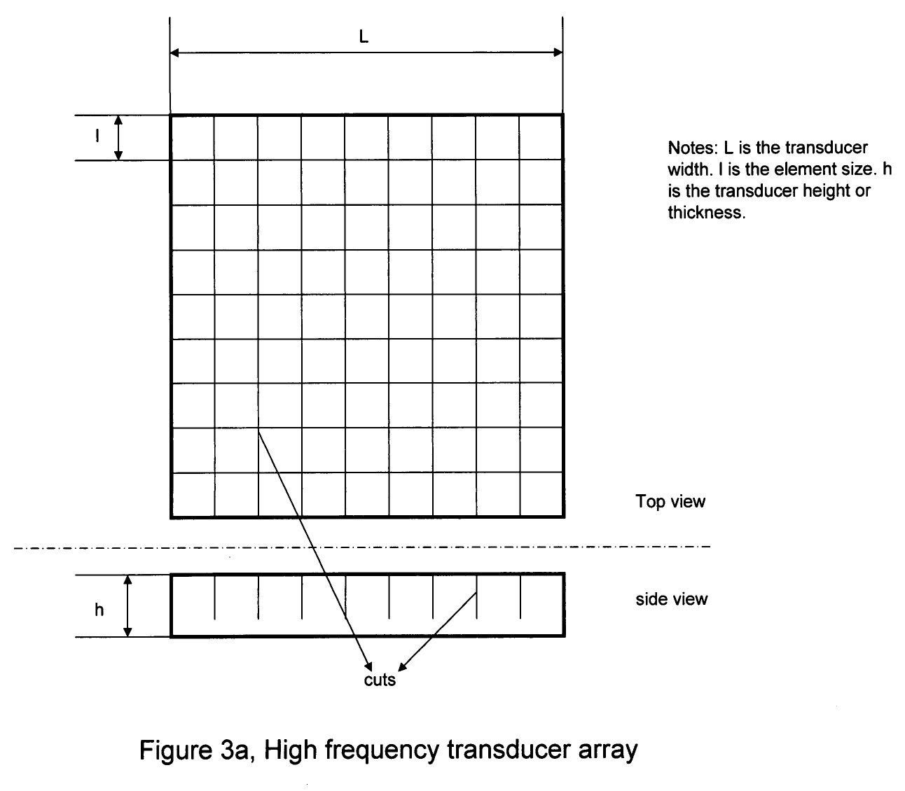

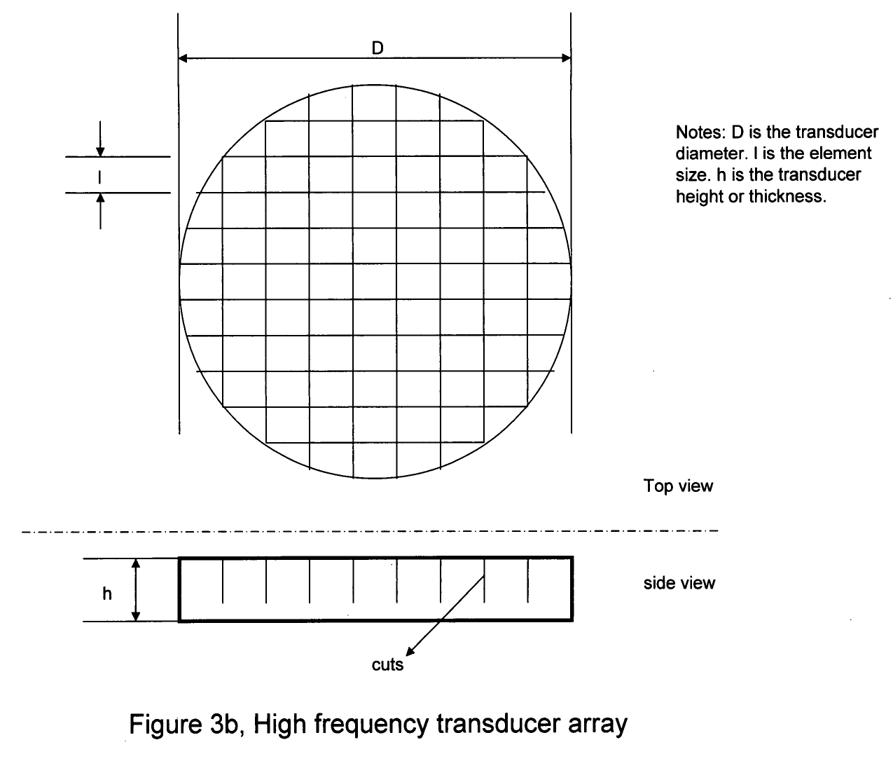

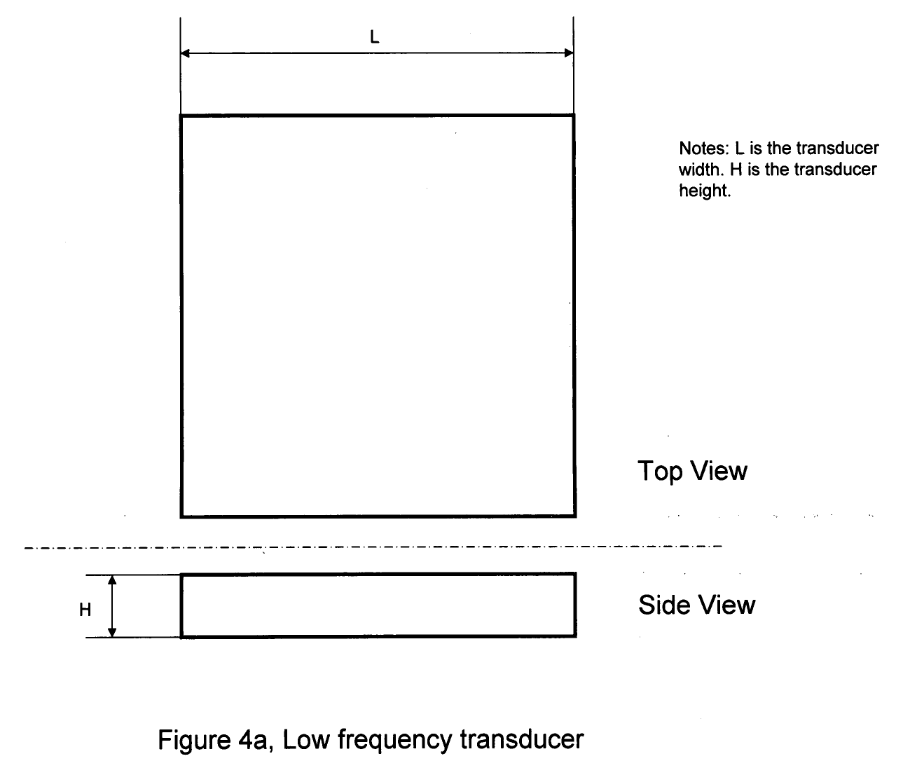

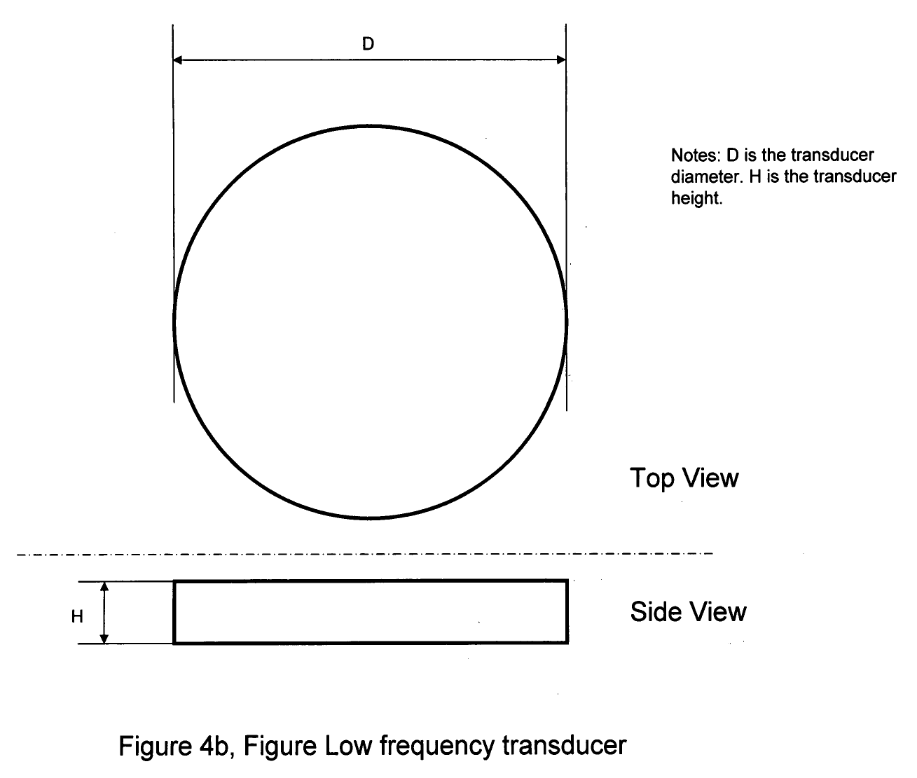

High frequency and high energy ultrasound beams come out from transducer Unit 9 (PZT-HF) in FIG. 2. Unit 9 has at least one transducer array, which has square or circular shapes in FIGS. 3 and 4. Or it can have any convenient shape that fits the design of probe. The transducer array can be flat or slightly curved. The transducer array is composed of multiple transducer elements, which can share common ground or power on one side. On the other side, the elements are separately connected to its own driving circuit. Each element has its own driving signal. The driving signals are phase-coordinated to focus ultrasound at a certain position.

The transducer, Unit 9 is placed outside the human body. To reduce the ultrasound loss, some kind of gel shall be applied to the skin to reduce the mismatch of ultrasonic impedance. The ultrasound beams pass the liquid layer, the gel and the skin, and reach the target adipose tissue. The ultrasound will keep penetrating the human with attenuation. Each ultrasonic beam comes from the element of transducer array. These ultrasonic beams are phase-coordinated by signals from a very accurate timing circuit.

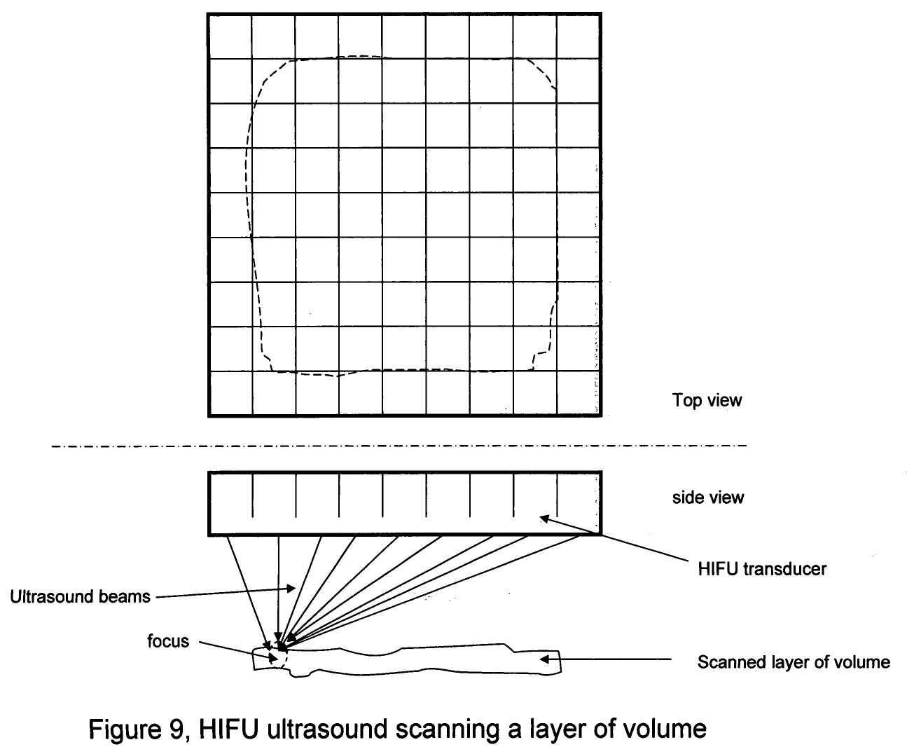

The size of element of transducer arrays in Unit 9 is analytically calculated to obtain certain ratio against the emitted ultrasound wavelength. At the right ratio the majority of acoustic (or ultrasonic) energy is confined in a certain volume at the certain distance from the transducer. Once the major ultrasonic energy of ultrasound beams from all the elements is confined within a volume, the ultrasound beams from the elements can be phase-coordinated so that the total acoustic pressure reaches maximal value at a certain spot in the target volume. This spot of maximal pressure value is called acoustic focus. The focus can move along on a layer in the target volume by changing the phase delays among the ultrasound beams. The varying acoustic focus moves over a layer or focused plane in the target volume is called scanning of layer of volume. The scanning is shown in FIG. 9. A square shape of 2-dimensional transducer array is used to illustrate the scanning process. The scanning process will let the scanned adipose tissue to have a chance to cool down. And most important the scanning ultrasound will push and pull the adipose cells, and eventually cause them to disrupt.

The focused ultrasonic exposure level is well controlled so that other characteristic tissues will not be damaged.

The focused ultrasound has a frequency of range from 250 Hz to 1.2 MHz. Generally the adipose tissue shall have a continuous characteristic acoustic receiving spectrum. But because of inter-person variations, there is only a statistical spectrum. The receiving spectrum shall be a function of cellular sizes. But the adipose cells change their size dramatically. Therefore in general, the lower frequency of ultrasound shall have longer treating time. The higher frequency of ultrasound shall have shorter treating time.

A driving circuit of high-frequency ultrasound 6 or Unit 6 is to drive high-frequency ultrasound transducer. Since each element of the transducer array has to be driven individually, the driving circuitry is an array of some identical circuit. But these identical circuits have different inputs and controls.

A hardware timing circuit 3 or Unit 3 generates timings for the ultrasound driving circuitry to focus the ultrasound beams, and to vary the ultrasound focus to scan a layer of biological tissue. Since the elements of the transducer arrays may have some frequency variations, each element can be calibrated and driven at its own oscillating frequency. This will take maximal ultrasonic energy out from the transducers. This timing circuit 3 can generate different frequencies of input signals for each driving circuit.

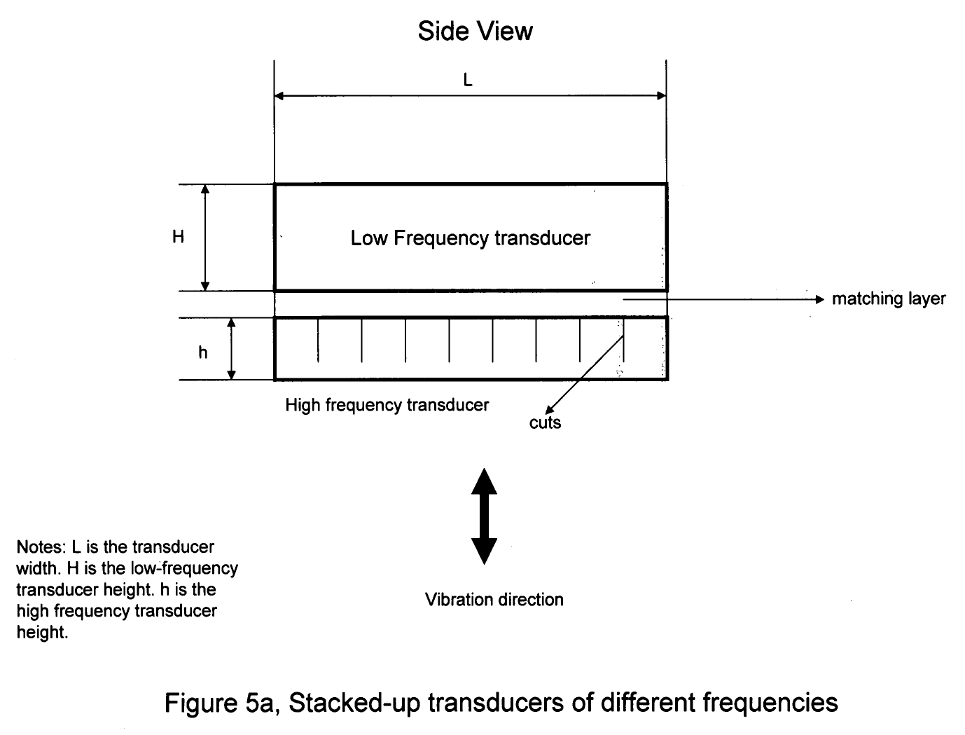

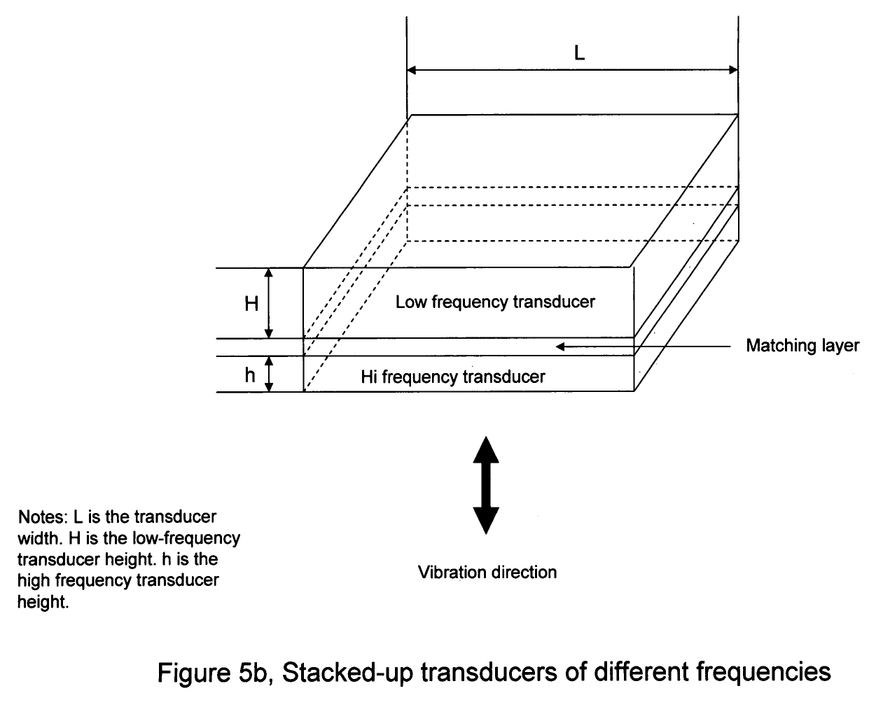

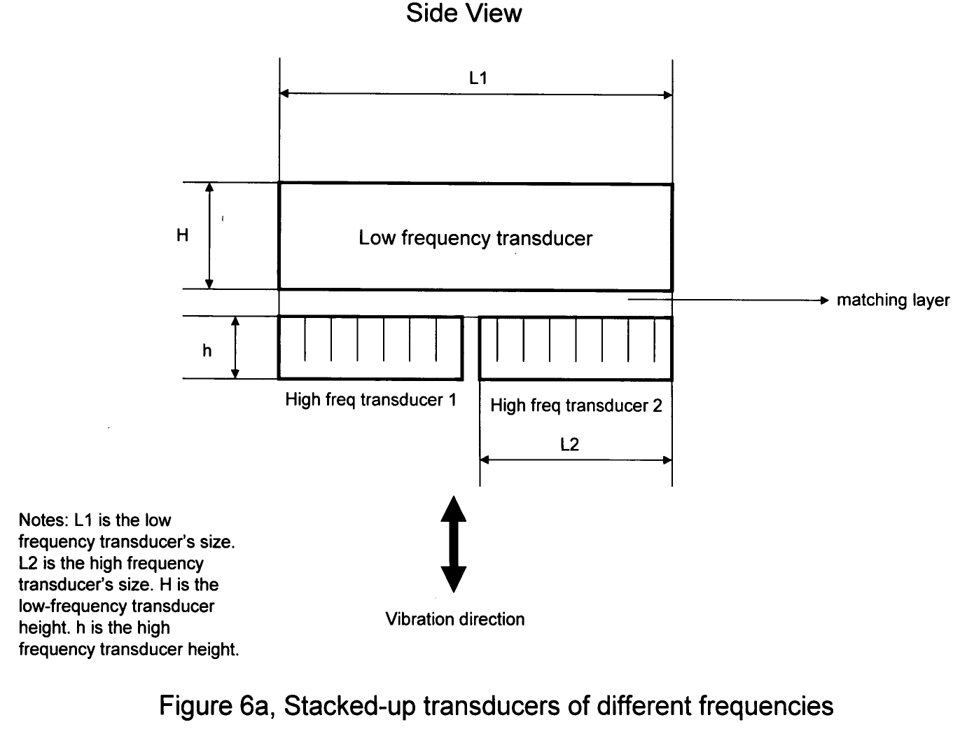

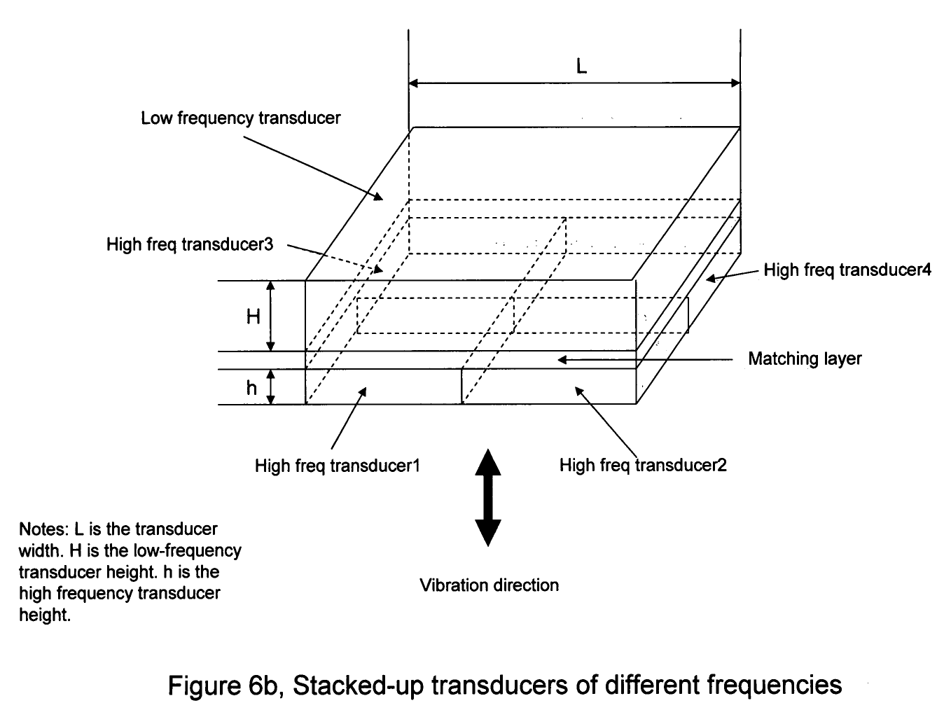

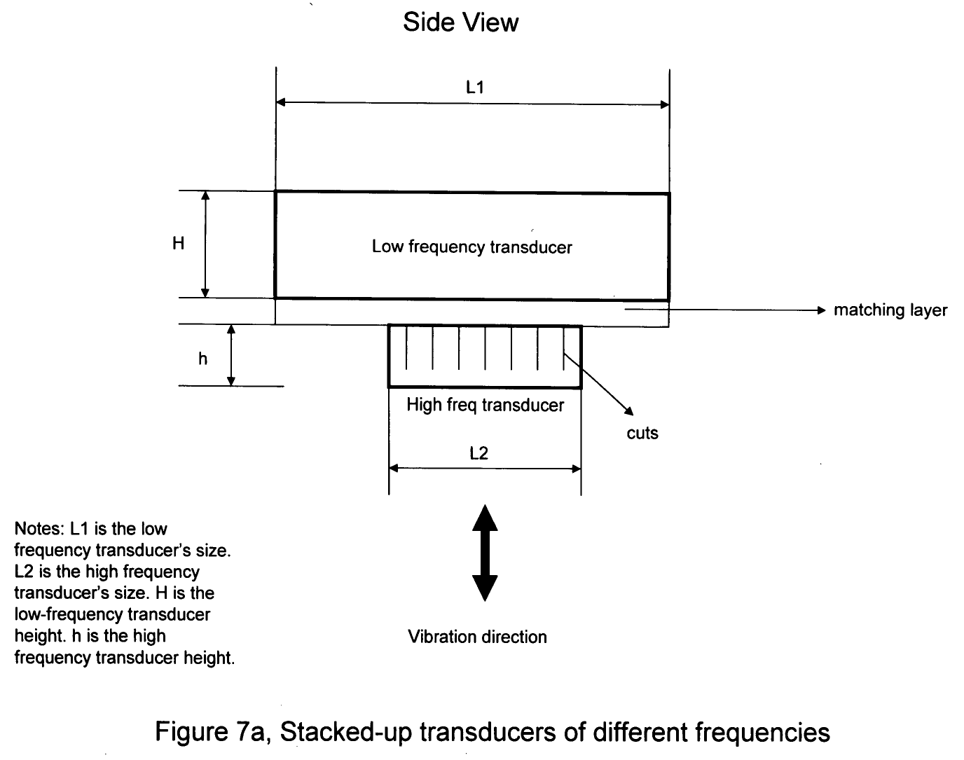

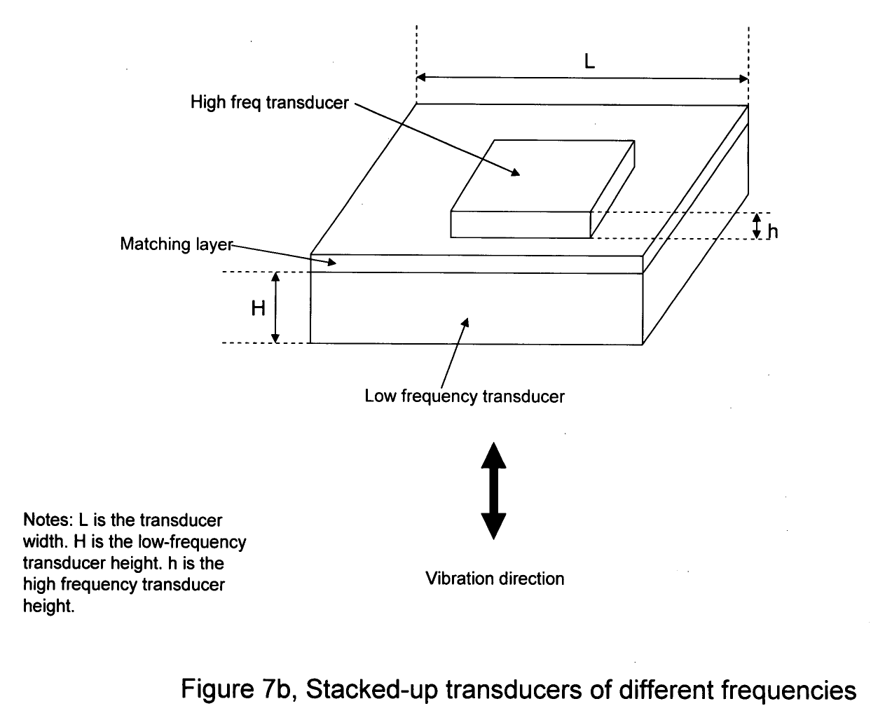

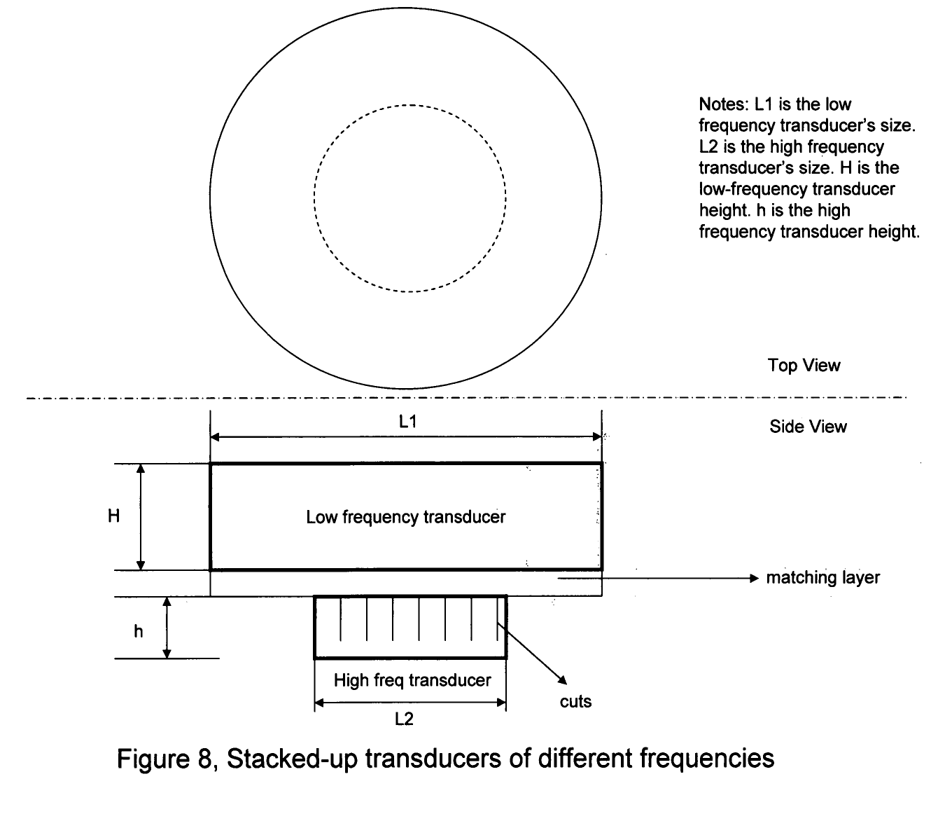

When the scanned adipose cells are broken, they shall be brought into lymphatic circulation as soon as possible and as much as possible. It has been proved that low-frequency ultrasound has vasodilatation effect. An ultrasound transducer Unit 8 (PZT L-F) placed outside the human body generates unfocused low-frequency and high-energy ultrasound, which will penetrate the skin and reach the target adipose tissue. It dilates the local blood vessels within and around the target volume. This will help taking the broken adipose cellular debris into lymphatic circulation. The transducer Unit 8 can have square or circular shapes like the ones in FIGS. 5 and 6, or any convenient shape with a definite frequency.

During each session of scanning, the level of ultrasound energy is constant. Multiple scanning sessions may have different energy levels. The energy level control is intelligent so that it can handle inter-personal variations.

There can be multiple ultrasonic focuses to scan at least one layer of volume. The variable ultrasonic focuses are selectively breaking the scanned adipose tissue, and are not breaking non-adipose tissues.

A driving circuit of low-frequency ultrasound, Unit 5 (L-F PZT Driving Circuit) is to drive low-frequency ultrasound transducer. Unit 5 receives timing from the hardware timing circuit 3.

A power supply of ultrasound transducer 7 or Unit 7 provides power to the transducers of both low-frequency and high-frequency. The power level is programmable. This is to accommodate the inter-person ultrasound variations and inter-stage variations of breaking adipose tissue. Unit 7 has an interface to the control hardware 4 wherein the driving power level can be set.

The invention also employs a digital signal processing (DSP) sub-system 2 or Unit 2. Unit 2 is the central control of this ultrasound system. It generates the parameters of timings for the timing hardware circuitry. It controls the ultrasound energy level. It analyzes the quantified information of backscattered ultrasound from AD Unit 11 in FIG. 2. It maps the quantitative variations of acquired information to quantity of broken adipose tissue. It monitors all system components.

The invention uses a novel way to control the process of breaking the target adipose tissue. It employs a feedback mechanism to tell the central control subsystem and operators the on-going procedure. The feed back information comes from a backscattered ultrasound receiver 10 or Unit 10 in FIG. 2. The ultrasound backscattered from the scanned layer of tissue is amplified, digitized, and analyzed periodically to detect the state changes of the target adipose tissue.

The invention uses quantization of backscattered ultrasound to get the feedback information. When the incident ultrasound hit adipocytes it scatters back. The ultrasound backscatters is related to the density of fat cells and the ratio of cell size and ultrasound wavelength. Unfortunately there is no analytic solution for solving their relationship. There are only statistical solutions. By measuring this backscattered ultrasound, we can monitor the ultrasonic breaking process of adipose tissues. During the ultrasonic breaking process, the physiological characteristics of adipocytes will change the backscattered ultrasound. Some variables of information are derived from the quantized backscattered ultrasound to detect and measure the changes of broken adipocytes.

Target values of the variables can also be set to stop the said ultrasonic breaking process. In this way, we can control what percentage of fat cells to be broken. The system can use multiple receiving piezoelectric sensors to measure the backscatter changes to boost the accuracy.

The quantitative information can also boost the effectiveness of breaking adipose tissue. It resolves the problems associated with inter-personal variations. Because of ultrasonic inter-person variations the ultrasonic exposure of breaking fat cells is different from person to person. With the quantitative information, different people will get different treatment under the safety limit.

A hardware power control circuit 4 or Unit 4 is mainly an interface between the DSP subsystem 2 and PZT power circuit 7. It interprets the DSP subsystem 2's power control info to control the power level.

An analog-to-digital (AD) converter 11 or Unit 11 in FIG. 2 converts the amplified backscattered ultrasound to a digital signal and sends it to the signal processing sub-system 2.

Any biological tissue in the volume not scanned by the focused ultrasound beams is safe and is not broken by the ultrasound energy.

It will be obvious to persons skilled in the art that variations or smart modifications can be made in the present invention without changing the fundamentals and coverage of the invention. Therefore it is intentional that the present invention covers any kind of variation and modification made from this invention since they fall into the scope of the stated claims and their equivalents.

1. An apparatus for using ultrasound to break adipose tissue comprising the steps of: (a) projecting ultrasonic energy at a target volume containing both adipose tissue and non-adipose biological tissues within a human body, wherein an ultrasonic focus is produced; (b) making said ultrasonic focus scan a layer in a target volume with a pre-set ultrasound power; (c) breaking the adipose tissue selectively in said scanned layer of said target volume; (d) controlling exposure of said ultrasound, wherein said exposure does not break non-adipose tissues in said layer of volume; (e) controlling said ultrasonic focus, wherein focused ultrasound does not break biological tissues outside said scanned layer of target volume.

2. An apparatus for breaking adipose tissue according to claim 1, further comprising the step of using a first ultrasound transducer arrays to deliver ultrasound energy to said target volumes wherein said ultrasound transducer arrays are spatially and phase-coordinated to obtain said ultrasonic focus in said target volume, wherein said ultrasonic focus is a maximal summation of ultrasonic pressure at an instantaneous spatial spot.

3. An apparatus for breaking adipose tissue according to claim 2, further comprising the step of generating one or a plurality of ultrasonic focuses.

4. Apparatus for breaking adipose tissue according to claim 3, wherein said ultrasonic focus can vary in any spatial pattern in the said target volume to scan one or a plurality of layers of biological tissues in said human body.

5. An apparatus for breaking adipose tissue according to claim 3, further comprising the step of adjusting
a distance of at least one said scanned layer of target volume from said ultrasound transducer arrays
and
a thickness of at least one said scanned layer in target volume.

6. An apparatus for breaking adipose tissue according to claim 1, where said focused ultrasound has a frequency range between 250 KHz and 1.2 MHz.

7. An apparatus for using ultrasound to break adipose tissue comprising the following steps of: (a) sensing a cavitation in said layer of target volume and feeding back quantitative information to a central computing system; (b) monitoring said quantitative feedback information about said cavitation of said target layer of volume; (c) performing quantitative adipose breaking using said quantitative feedback information.

8. An apparatus for breaking adipose tissue according to claim 7, further comprising the step of delivering a low-energy ultrasound to said target volume from either a second ultrasound transducer array or said first transducer array.

9. An apparatus for breaking adipose tissue according to claim 8, further comprising the step of detecting the reflection of said low-energy ultrasound backscattered from said target layer of volume using either said first ultrasound transducer array or said second ultrasound transducer array.

10. An apparatus for breaking adipose tissue according to claim 9, further comprising the step of digitizing said ultrasound backscattered from said target volume.

11. An apparatus for breaking adipose tissue according to claim 10, further comprising the step of extracting and quantizing said information from the digitized backscattered ultrasound.

12. An apparatus for breaking adipose tissue according to claim 11, further comprising the step of expressing the said information in at least one of time, frequency, space, and energy domains, or a combination of said domains.

13. An apparatus for breaking adipose tissue according to claim 11, further comprising the step of expressing said information in any transformed mathematical domain from at least one of time, frequency, space and energy domains, or a combination of said domains.

14. An apparatus for breaking adipose tissue according to claim 11, further comprising the step of calibrating said quantitative feedback information to a quantitative amount of broken adipose tissue or a percentage of broken adipose tissue in the target volume.

15. An apparatus for breaking adipose tissue according to claim 7, further comprising the step of
Monitoring the variations of said extracted information before and during the adipose tissue breaking process;
Obtaining a first set of quantitative values of said information of multiple domains before said adipose breaking process as baseline values;
Comparing a second set of quantitative values of said information against said baseline values during the adipose breaking process.

16. An apparatus for breaking adipose tissue according to claim 7, further comprising the step of mapping quantitative variations of said extracted information to a quantity of broken adipose tissue.

17. An apparatus for breaking adipose tissue according to claim 16, wherein said quantitative variations are mapped to an absolute amount of broken adipose tissue or a percentage of broken adipose tissue in said target layer of volume.

18. An apparatus for using ultrasound to break adipose tissue according to claim 1, further comprising the following steps of delivering a second frequency of ultrasound at said scanned layer of target volume to cause said broken adipose tissue to go into the body circulation.

19. An apparatus according to claim 18, further comprising the step of using at least one different ultrasound transducer to deliver the said second frequency of ultrasound to said target layer of volume to increase blood flow to the said volume and to increase the lymphatic flow to take the broken adipose tissue away from said volume into body circulation.

20. An apparatus according to claim 18, where the said second frequency of ultrasound has a frequency range between 28 KHz and 250 KHz.

21. An apparatus according to claim 18, further comprising the step of bundling a plurality of ultrasound transducers of different frequencies in a layered manner wherein the ultrasound transducers of the same frequency stay in the same layer.

22. An apparatus according to claim 18, further comprising the step of having one or a plurality of coupling layers between the said ultrasound transducers, wherein said coupling layers bond said transducers and minimize an ultrasonic loss when the ultrasound goes through said different layers.
